(19) United States
(12) Patent Application Publication
(10) Pub. No.: US 2014/0309276 A1
(43) Pub. Date: Oct. 16, 2014

(54) Pharmaceutical Composition Comprising a Mixture of Carboxylated Oligonucleotides

(71) Applicants: Artur Martynov, Kharkov (UA); Boris S. Farber, Brooklyn, NY (US); Sonya Sophya Farber, New York, NY (US)

(72) Inventors: Artur Martynov, Kharkov (UA); Boris S. Farber, Brooklyn, NY (US); Sonya Sophya Farber, New York, NY (US)

(21) Appl. No.: 13/861,624

(22) Filed: Apr. 12, 2013

(51) Int. Cl. A61K 31/7088

(52) U.S. Cl. CPC A61K 31/7088
USPC 514/44 A

(57) ABSTRACT

The invention describes a pharmaceutical composition, comprising a mixture of carboxylated oligonucleotides, obtained by hydrolysis of natural polynucleotides, that results in oligonucleotide mixture, and carboxylation of purine nucleotide bases in the oligonucleotide mixture. The oligonucleotide mixture (DNA, RNA, or a combination of the two) of plant, animal, or fungal origin is conducted through chemical or biochemical enzymatic procedures, and then, a chemical modification of the oligonucleotides is provided, with carboxylation of their purine nucleotide bases through alkylation with monochloroacetic acid, or acylation with succinic anhydride. The pharmaceutical composition may be used in humans and animals for cancer treatment, immunodeficiency, viral infection diseases, etc.

### CROSS REFERENCE TO RELATED APPLICATIONS

The present application is a continuation-in-part of the application Ser. No. 12/931,468, filed Feb. 1, 2011, which is a continuation of the International Application No. PCT/RU2010/000691, filed Nov. 22, 2010.

### TECHNICAL FIELD

This invention relates to medicine and pharmaceuticals, specifically, to pharmaceutical compositions and methods of manufacturing pharmaceutical compositions.

### BACKGROUND OF THE INVENTION

The task of the invention was to develop the pharmaceutical composition of modified oligonucleotides with anticancer properties and the methods of producing them.

The given task is addressed through selection of oligonucleotide mixture produced by hydrolysis of polynucleotides, and modification of this oligonucleotide mixture by carboxylation of the nucleotide bases.

The given task is also addressed through the development of a pharmaceutical composition, comprising a mixture of carboxylated oligonucleotides, provided by hydrolysis of natural polynucleotides that resulted in oligonucleotide mixture, and carboxylation of purine nucleotide bases in the oligonucleotide mixture. The oligonucleotide mixture (DNA, RNA, or a combination of the two) of plant, animal, or fungal origin is obtained through chemical or biochemical enzymatic procedures, and then, a process of chemical modification of the oligonucleotides is provided, with a carboxylation of their purine nucleotide bases through alkylation with monochloroacetic acid, or acylation with succinic anhydride.

### TERMS

The term “pharmaceutical composition” refers to a mixture of carboxylated oligonucleotides produced by hydrolysis of a whole DNA, or RNA of eukaryotic cells, and subsequently carboxylated by acylation or alkylation of structure in purine nucleotide bases. Also, the composition may include additional substances permissible in pharmaceutical excipients, such as glycerol, PEG-400, preservatives, stabilizers, cryogenic and thermal protectors.

The use of one of the oligonucleotides in the mixture as an anticancer agent is not expected, since only the mixture of many oligonucleotides has the ability to form with each other and the target insoluble stable supramolecular assemblies.

Anti-cancer properties—refer to the direct cytotoxic, or cytostatic effect on cancer cells (not immunomodulating), or the synergistic cytotoxic, or cytostatic effect in interaction with classical cytotoxic anticancer drugs.

Hydrolysis refers to a process of DNA or RNA polymer degradation to smaller oligomer fragments—oligonucleotides, the degradation is catalyzed by nuclease enzymes, or by their synthetic analogs. Also, hydrolysis refers to acid hydrolysis of DNA or RNA polynucleotides through boiling them in mild conditions by classical methods (without complete destruction to mononucleotides). The average size of oligonucleotides with such hydrolysis ranges between 3 and 30 n.b. (nucleotide bases).

Natural polynucleotide refers to a whole DNA or RNA extracted from one or more species of eukaryotic organisms, such as yeast and plant cells, provided by classical methods; the whole DNA or RNA is ultimately purified of other non-nucleotide substances—peptides, lipids, polysaccharides and low molecular weight substances.

Carboxylation refers to introduction of carboxyl groups into the oligonucleotide structure through formation of a new covalent bond, or through acylation of dicarboxylic acids, or alkyl carboxylic acids (not halides) by anhydrides. In the case of using polycarboxylic acid anhydride the amide group forms with exocyclic amino group in the residues of adenine and guanine, and in the case of using alkyl carboxylic acids the alkylamines derivatives are formed regarding the endocyclic nitrogen of adenine and guanine.

Acylation involves replacing only part of the amino groups in the structure of the purine bases. Part of the purine bases remain not substituted. The substitution rate can be calculated based on the formulas of combinatorial mathematics to obtain the maximum number of combinations and substitutions, or empirically, through the synthesis of many derivatives with varying degrees of modification, and then, selection of the most pharmacologically active derivatives.

Purine nucleotide bases—reflect residues of adenine and guanine, which remain in oligonucleotides of the hydrolyzed DNA or RNA.

### SUMMARY OF INVENTION

A pharmaceutical composition, comprising a mixture of carboxylated oligonucleotides, obtained by hydrolysis of natural polynucleotides, that resulted in a oligonucleotide mixture, and carboxylation of purine nucleotide bases in the oligonucleotide mixture.

Hydrolysis of polynucleotides is provided with natural or synthetic nucleases—ribonucleases or deoxyribonucleases. Also for this purpose, acid or alkaline hydrolysis can be used by mineral acid or alkaline, which results in olygonucleotide mixture. Next, the olygonucleotide mixture is modified through acylation of the amino groups in the structure of oligonucleotides, or through their alkylation by halogen-carboxylic acids. For acylation of amino groups in oligonucleotide structure succinic anhydride can be used. For alkylation of amino groups in oligonucleotide structure monochloroacetic acid is used.

This drug has a wide spectrum of activity and a low level of toxicity; it is suitable for industrial production and is effective at any stage of cancer.

### DESCRIPTION OF THE DRAWINGS

FIG. 1 illustrates the reaction of adeninozin (I) with succinic anhydride (III), and monochloroacetic acid (II) with the formation of succinamide of adenosine (V) and carboxymethylguanosine (IV), respectively.

FIG. 2 illustrates the reaction of guanosine (la) with monochloroacetic acid (II) and succinic anhydride (III) with the formation of carboxymethylguanosine (IVa) and succinamidaguanosine (Va)

FIG. 3 illustrates the visualization of results of mixing CfVr+CfIFar in the first cavity, Vr+CfIFar in the second cavity, IFar+CfVr in the third cavity, Vr+CfVr in the fourth cavity, IFar+CfIFar in the fifth cavity, and unmodified RNA together Vr+IFar in the sixth cavity at using gel electrophoresis in phosphate buffer under standard conditions with visualization in U/V light after treatment with ethidium bromide.

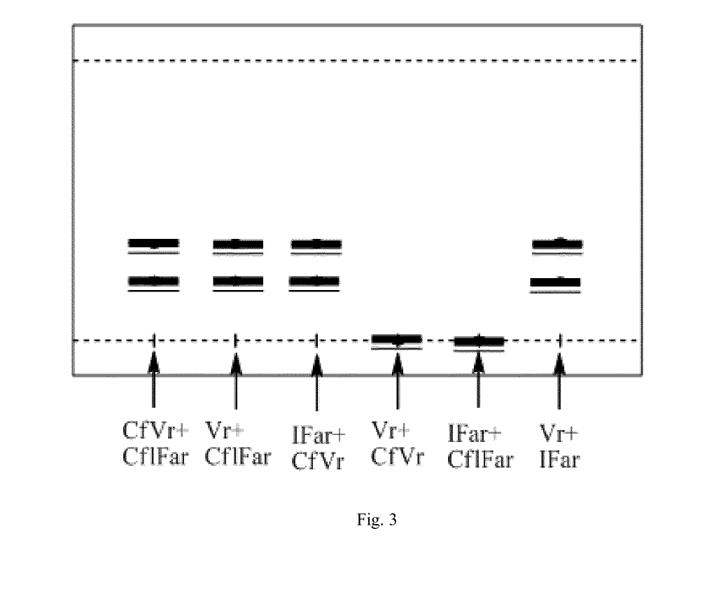

Only homogeneous RNA reacted with their modified derivatives and remained at the starting line in the insoluble form. IFar molecule is substantially heavier and remains close to the start and forms the first lane, and Vr creates a second lane in the area of light molecules.

### DETAILED DESCRIPTION OF THE INVENTION

The specific inhibitor of valine t-RNA is developed that has powerful anti-cancer properties. Inhibition of t-RNA in a cell leads to stop in protein synthesis and thus, to cell destruction. Thus, the first step is to choose a target, namely, valine t-RNA. The structure of this RNA is well researched and presented in many databases. It is also well known, that this RNA has 18 residues of adenine in its structure.

These adenines are attacked by acylation agents in the cold water medium. In order to obtain from a single t-RNA molecule, via modification, thousands of fragments with the ability of structure self-organization, it is necessary to calculate the amount of the modifier and the number of adenines, which must be modified in accordance with the combinatorial principles and combinatorial chemistry, according to the formulas 1 and 2 below.

As a result of calculations, we have m=262,143 and k=2359296, which provides the molar ratio of t-RNA molecules to anhydride as 1:9. As a result of combinatorial modification of the whole t-RNA molecule in this mole ratio, there are produced 262,143 different molecules of acylated t-RNA derivatives. These molecules are able to react with each other and form complex supramolecular assemblies, find targets, and include them in their structure with complete inactivation of these targets.

In fact, a system with such variety of elements in structure behaves as a quasi-living self-organizing system that can actively respond to external factors and reorganize its supramolecular structure.

If prior to its modification t-RNA is fragmented by ribonuclease, then, first, four oligomer fragments are formed, and the number of different molecules after adenine acylation increases. Small size of t-RNA oligomer fragments allow them to easily penetrate the tissues and cells and to inactivate t-RNA targets there. The fragmentation of biopolymer to oligomer fragments is needed to obtain good bioavailability of the drug.

If more than one t-RNA, but all RNA from cell are used, for example, in yeast cell, the number of different elements in the system would be close to a billion, and such a mixture of oligomer fragments would act as a living system, performing its function—i.e., finding complementary parts in the original RNA and conjugate with them (Antican Preparation). The method of producing the pharmaceutical composition, comprising a mixture of carboxylated oligonucleotides, includes the following steps:
determination of a quantity of nitrogen-containing groups that are positively charged and available for modification in the biopolymer target (of DNA, RNA, or their mixture) by identification of adenine and guanine quantity after hydrolysis with the HPLC method;
determination of a modification rate for the determined quantity of adenine and guanine groups;
fragmentation of the biopolymer target into oligomer fragments;
combinatorial modification of the oligomer fragments by substituting a selected number of adenine and guanine for negatively charged groups, the number is selected according to the modification rate (according to formulas 1 and2);
application of the modified oligomer fragments as a self-assembled drug that is complementary for the biopolymer target

### EXAMPLE 1

1 g (Mo) mixture of RNA from yeast is taken that is produced by the classical method as shown in [O. Matte, C. Chabalier, R. Ratomahenina, Jean-Pierre Bossy, Pierre Galzy. Isolation and characterization of a RNA-virus like particle from Candida curvata. Biology of the Cell, Vol. 68, Issue 2, pages 159-162, 1990], then it is dissolved in 100 ml of 0.4 N NaOH, and hydrolyzed as described in [Cox R. A., Gould H. J., Kanagalingam K. A study of the alkaline hydrolysis of fractionated reticulocyte ribosomal ribonucleic acid and its relevance to secondary structure. Biochem J. 1968 February; 106(3): 733-741].

The solution is then cooled; 0.1 ml of it is selected, and the quantitative composition of each mononucleotide is determined by the HPLC method. The following chromatography conditions are used: sample is passed through a chromatographic column of Nucleosil-18t type, using an ultraviolet detector at 260 nm. The column was calibrated with 10-fold dilutions of ATP. In the chromatogram four absorption lanes are produced that are characteristic of each mononucleosis product.

The peak area is proportional to the absorption amount (weight) of each mononucleosis product, calculations are carried out automatically with the conversion into the number of moles by chromatograph processor in a classic way. As a result of the conversion of the amount of each nucleoside in 0.1 g weighed portion of the whole (* 10) is produced the mass of each nucleoside (ma, mg, mu, mc) in weighed portion (Mo). The calculations are provided of the total number of groups (n) available for modification in the initial weighed portion of the modifier (Mm).

These calculations are needed at the output of the carboxylation reaction (acylation or alkylation) to get the maximum variety of derivatives with respect to the sites of substitution in adenine residues (I) and guanine (la). Calculations are performed using the following formulas:

$$Mm = {M}_{r}^{m} ⌊ \frac{n {2}^{( n - 1 )}}{( {2}^{n} - 1 )} ⌋ , \qquad ( 1 )$$

where
n—Quantity of the purine bases, available for modification of the taken weighed portion of dry oligonucleotide composition,
Mᵣ^{m}—molecular weight of the modifier, g/mol
n—is calculated by the formula:

$$n = ( \frac{{M}_{o} - {m}_{u} - {m}_{c} - {m}_{a}}{{M}_{r}^{G}} ) + ( \frac{{M}_{o} - {m}_{u} - {m}_{c} - {m}_{g}}{{M}_{r}^{A}} ) , \qquad ( 2 )$$

where
Mo—portion weight of the dry mixture of oligonucleotides after hydrolysis, g;
mu—uridil weight in the portion of the dry mixture of oligonucleotides after hydrolysis, identified by chromatography after complete hydrolysis, g;
mc—cytosil weight in the portion of the dry mixture of oligonucleotides after hydrolysis, identified by chromatography after complete hydrolysis, g;
ma—adenosyl weight in the portion of the dry mixture of oligonucleotides after hydrolysis, identified by chromatography after complete hydrolysis, g;
mg—guanozil weight in the portion of the dry mixture of oligonucleotides after hydrolysis, identified by chromatography after complete hydrolysis, g;
M_{T}^{T}—molecular weight of guanozil g/mol;
M_{T}^{A}—molecular weight of adenosyl g/mol;

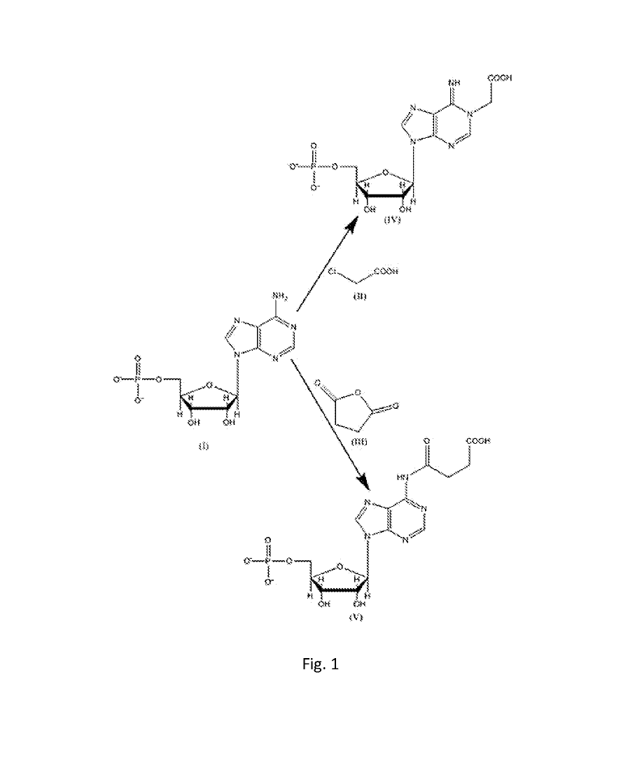

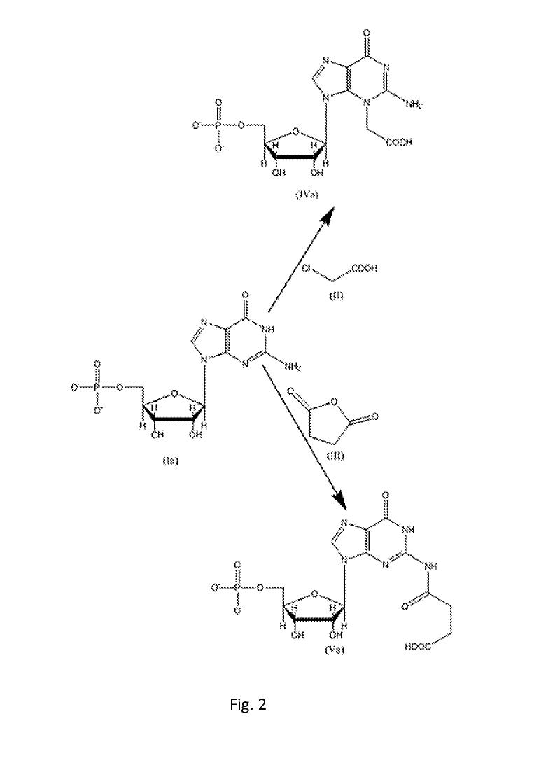

The modification is conducted (FIGS. 1, 2) by adding the calculated amount of modifier [succinic anhydride (III) or monochloroacetic acid (II)) in the amount of (Mm) and boiling with reflux condenser at pH 8.0 for 15 minutes.

For (IV): 1H NMR (CD3)2SO, 400 MHz) for (IV): 3.58 (s, 2H), 3.95-4.28 (broad s, 5H), 11.00 (s, 1H), for (IVa): 3.58 (s, 2H), 3.95-4.28 (broad s, 5H), 8.56 (s, 2H) 11,3 (s, 1H), 12.00 (s,2H); for (V): 2.49 (d, 2H), 2.70 (d, 2H), 3.58 (s, 2H), 4.00-4.75 (broad s, 6H), 8.35 (s, 1H), 8.60 (s, 1H), 9.15 (s, 1H), 11,1 (s, 1H), 12.00 (s, 2H); for (Va):): 2.49 (d, 2H), 2.71 (d, 2H), 3.57 (s, 2H), 4.03-4.75 (broad s, 6H), 8.02 (s, 1H), 7.98 (s, 1H), 11,3 (s, 1H), 11.98 (s, 2H).

The synthesis of derivatives of the mixture of oligonucleotides at the level of modified mononucleosis products is presented in FIGS. 1 and 2.

Replaced are only some of the available groups with the formation of thousands of derivative oligonucleotides reacting with each other, and forming complex supramolecular systems. Such systems have the lowest energy only during the neutralization of the carboxyl groups by amino groups.

Accordingly, when a target appears—i.e., the original intact unmodified RNA molecule (DNA), the formation of insoluble supramolecular complex is observed as a result of deposition of carboxylated fragments of oligonucleotides on the whole original target due to complementarity of carboxyl and amino groups. This mechanism allows to inactivate the original target and such a system manifests significant anti-cancer properties.

Anti-cancer properties of the system are related to the inactivation of most of the cellular RNA in cancerous tumors through their conjugation with fragments of carboxylated oligonucleotides. The specificity of the self-assembly of carboxylated oligonucleotide fragments of RNA in whole RNA molecule is confirmed on the model of valine RNA (Vr) and RNA of alpha-interferon (IFar): carboxylated valine RNA fragments (CfVr) do not lead to the formation of insoluble supramolecular complex with IFar, and only the addition of the original solid valine RNA allows the formation of the insoluble supramolecular systems.

Similarly had been executed an experiment with carboxylated RNA fragments of alpha-interferon (CfIFar), which reacted only with the original RNA of interferon-alpha (IFar) and did not react with valine DNA. Supramolecular complexes of the original RNA and carboxylated fragments are black oily substances that are not soluble in any solvent, and are destroyed only by solutions of a strong alkali. The formation of these complexes is very fast with condensation of deposits not at the bottom of the tube, but on the walls in the form of oily droplets.

Due to the complete insolubility and lack of crystallization ability in these systems, it is not possible to provide their chromatographic or X-ray structure analysis at the present stage of development of physical and chemical methods. Homologous RNA with its modified derivative is at the starting line when using electrophoresis for visualization.

### EXAMPLE 2

### Obtaining a Modified Oligonucleotides Composition

The microbial biomass contains up to 11% nucleic acids and may serve as a raw material for the obtainment of microbial RNA, on the basis of which it is possible to produce derivatives that are a mixture of oligonucleotides. The object of study for this stage of the work was RNA taken from Saccharomyces cerevisiae yeast biomass.

Separation of summary RNA from Baker's Yeast. Four kg of compressed yeast was thawed at room temperature. It was ground and suspended in 8 L of boiling water containing 300 g of sodium dodecal sulfate. The suspension was boiled for 40 minutes while being stirred constantly. It was then poured into steel centrifuge cups, which were quickly placed in ice and cooled to 5° C. (˜15 min.), after which they were centrifuged in a 6K15 German-made centrifuge (17000 g, 10 min, 4° C.). The sediment and a part of the gel-like interphase were removed, and the RNA that had migrated to the supernatant was separated out. This was accomplished by adding NaCl to the supernatant obtained in the previous stage, until a final concentration of 3 M was reached. After dissolving the salt, the suspension was left for 1 hour to allow the formation of sediment, which was then separated by a centrifuge at 17000 g over 10 min. The sediment was rinsed with two portions of 8 l 3 M NaCl each, suspended in 2 l of ethanol, and left overnight. The next day, the suspension was centrifuged at 17000 g over the course of 10 minutes. The sediment was dissolved in distilled water to an RNA concentration of 450 D260 U/ml (˜1.6 l). The solution was clarified by centrifuging under the same conditions, after which the RNA was precipitated from the supernatant through adding NaCl to a final concentration of 0.15 M and an equal volume of ethanol (˜2 l). The sediment formed was separated from the supernatant through centrifuging (17000 g, 10 min), cleaned in 1 l of ethanol, and dehydrated in a CaCl vacuum desiccator. All operations were conducted at a temperature of 0-4° C.

Separation of High-Polymer RNA from Baker's Yeast

Day 1: RNA Extraction.

7.5 g of sodium dodecal sulfate were dissolved in 300 ml of water in a heat-resistant one-liter glass beaker. The solution was brought to a boil, and over a period of 5 minutes, 30 g of dry yeast were added in such a manner that the temperature of the suspension did not fall below 98° C. The suspension obtained was boiled for 40 minutes while being stirred constantly, and boiling water was added to keep the mixture at 300 ml as the original water boiled off. At the end of the extraction, the suspension was cooled to approximately 60° C. Freshly boiled water was added to a volume of 450 ml. This was mixed, poured into a 500-ml measuring cylinder, and left to settle at a temperature of 20° C. for 22 hours.

Day 2: RNA Desalting and Rinsing the Desalted RNA with 3 M NaCl.

The cooled supernatant (280 ml) was transferred to a measured glass beaker with a volume of 500 ml. 81 g of NaCl was added to it, as was water, to make a total of 450 ml. This was mixed and left to settle at a temperature of 19° C. for 6 hours. After 6 hours, the interphase formed (250 ml) was bled off, and to the desalted RNA that was divided into two fractions (one part deposit, one part supernatant) was added 3 M NaCl up to 450 ml. This was mixed and left to settle overnight at a temperature of 19° C.

Day 3: Rinsing the Desalted RNA with 3 M NaCl.

The interphase (300 ml) was bled off, and 3 M NaCl was added to the desalted RNA up to 450 ml. This was mixed and left to settle at a temperature of 19° C. for 5 hours. After 5 hours, the interphase (280 ml) was bled off, and 3 M NaCl was added to the desalted RNA up to 450 ml. This was mixed and left to settle at a temperature of 19° C. for 3 hours. After 3 hours, the interphase (250 ml) was bled off, and 3 M NaCl was added to the desalted RNA up to 450 ml; this was mixed and left to settle overnight at a temperature of 19° C.

Day 4: Rinsing the Desalted RNA with Alcohol.

There is almost no top layer. Sediment occupies a volume of ˜100 ml. The supernatant was removed, and ethanol was added to the sediment up to a volume of 300 ml. This was mixed and left to settle at a temperature of 18° C. for five hours. After five hours, the supernatant was poured off, and a fresh portion of ethanol was added to the 140 ml of sediment up to a volume of 320 ml. This was mixed and left to settle at a temperature of 19° C. for 40 minutes, after which the supernatant was poured off. 120 ml of fresh ethanol was added to the 120 ml of sediment. This was mixed, and the supernatant was poured off after the mixture had been left to stand for 30 min. To the sediment (110 ml) was added 110 ml of fresh ethanol. This was mixed, and after 30 minutes of settling, the supernatant was poured off, and the RNA suspension was poured in a thin layer into a flat pan and air-dried at room temperature until the odor of alcohol had disappeared. The pure high-polymer RNA from the intermediate product was removed through water extraction in a cellophane dialysis bag.

Enzymatic Hydrolysis of RNA Polynucleotides

For a nuclease, the pancreatic ribonuclease (RNAase) had been selected with an activity of 14000 U/mg in a quantity of 0.4% of the mass of the RNA. The RNA hydrolysis was conducted over a period of four hours. The hydrolysis product was dehydrated in a flash drier.

Chemical modification of the hydrolysis product's oligonucleotides. A 3.5% water solution of the hydrolysis product was obtained; succinic anhydride was added in a quantity of 10-45% of the dry weight of the hydrolysis product; this was mixed in the cold until the anhydride was fully dissolved. The solution obtained was sterilized for 120 with flowing steam. The prepared solution was studied further for presence of anticancer properties.

The ability of the composition to inhibit mutagenesis in human endothelial cells is measured by their ability to inhibit 3H-thymidine incorporation into HUVE cells (human umbilical vein endothelial cells, Clonetics™). This assay has been well described in the literature (Waltenberger J et al. J. Biol. Chem. 269: 26988, 1994; Cao Yet al. J. Biol. Chem. 271: 3154, 1996). Briefly, 10″ cells are plated in collagen-coated 24-well plates and allowed to attach. Cells are re-fed in serum-free media, and 24 hours later are treated with various concentrations of composition (prepared in physiological solution, final concentration in the assay is 0.2% v/v), and 2-30 ng/ml VEGF165. During the last 3 hours of the 24 hour composition treatment, the cells are pulsed with 3H thymidine (NEN, 1 μCi per well). The media are then removed, and the cells washed extensively with ice-cold Hank's balanced salt solution, and then 2 times with ice cold trichloroacetic acid (10% v/v). The cells are lysed by the addition of 0.2 ml of 0.1 N NaOH, and the lysates transferred into scintillation vials. The wells are then washed with 0.2 ml of 0.1 N HCl, and this wash is then transferred to the vials. The extent of 3H thymidine incorporation is measured by scintillation counting. The ability of the composition to inhibit incorporation by 50%, relative to control (VEGF treatment with DMSO vehicle only) is reported as the IC50 value for the test compound.

Administration of the Composition of the present invention (hereinafter the “active compound(s)”) can be effected by any method that enables delivery of the compounds to the site of action These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration and the judgment of the prescribing physician However, an effective dosage is in the range of about 0 001 to about 100 mg per kg body weight per day, preferably about 1 to about 35 mg/kg/day, in single or divided doses for a 70 kg human, this would amount to about 0.05 to about 7 g/day, preferably about 0 2 to about 2 5 g/day In some instances, dosage levels below the lower limit of the aforesaid range may be more than adequate, while in other cases still larger doses may be employed without causing any harmful side effect, provided that such larger doses are first divided into several small doses for administration throughout the day.

The activity of the composition in vivo, can be determined by the amount of inhibition of tumor growth by a test compound relative to a control The tumor growth inhibitory effects of various compounds are measured according to the methods of Corbett T. H., et al “Tumor Induction Relationships in Development of Transplantable Cancers of the Colon in Mice for Chemotherapy Assays, with a Note on Carcinogen Structure”, Cancer Res, 35, 2434-2439 (1975) and Corbett, T H, et al, “A Mouse Colon-tumor Model for Experimental Therapy”, Cancer Chemother Rep (Part 2)”, 5, 169-186 (1975), with slight modifications Tumors are induced in the flank by s c injection of 1×106 log phase cultured tumor cells suspended in 0 1-0 2 ml PBS After sufficient time has elapsed for the tumors to become palpable (5-6 mm in diameter), the test animals (athymic mice) are treated with active composition (formulated by dissolution in appropriate diluent, for example water or 5% Gelucire™ 44/14 m PBS by the intraperitoneal (ip) or oral (po) routes of administration once or twice daily for 5-10 consecutive days In order to determine an anti-tumor effect, the tumor is measured in millimeters with Vernier calipers across two diameters and the tumor volume (mm3) is calculated using the formula Tumor weight=(length×[width]2)/2, according to the methods of Geran, R I, et al “Protocols for Screening Chemical Agents and Natural Products Against Animal Tumors and Other Biological Systems”, Third Edition, Cancer Chemother Rep, 3, 1-104 (1972) The flank site of tumor implantation provides reproducible dose/response effects for a variety of chemotherapeutic agents, and the method of measurement (tumor diameter) is a reliable method for assessing tumor growth rates

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository The pharmaceutical composition may be in unit dosage forms suitable for single administration of precise dosages The pharmaceutical composition will include a conventional pharmaceutical carrier or excipient and a compound according to the invention as an active ingredient In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions Such dosage forms can be suitably buffered, if desired Suitable pharmaceutical carriers include inert diluents or fillers, water and various organic solvents The pharmaceutical compositions may, if desired, contain additional ingredients such as flavorings, binders, excipients and the like Thus for oral administration, tablets containing various excipients, such as citric acid may be employed together with various dismtegrants such as starch, alginic acid and certain complex silicates and with binding agents such as sucrose, gelatin and acacia Additionally, lubricating agents such as magnesium stearate, sodium lauryl sulfate and talc are often useful for tableting purposes Solid compositions of a similar type may also be employed in soft and hard filled gelatin capsules Preferred materials, therefore, include lactose or milk sugar and high molecular weight polyethylene glycols When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art For examples, see Remington's Pharmaceutical Sciences, Mack Publishing Company, Easter, Pa., 15th Edition (1975).

Anticancer activity of Composition in a cell culture was made in a culture of HeLa-2 cells. For this purpose, 2-12 mcg of Composition per ml of medium were added to the 199 medium. A culture without Composition in it was used as a control. Cultures were observed daily over the course of five days. The Minimum Active Dose (MAD) of Composition was also considered to be the minimum amount of the drug that caused a degeneration of 90-95% of the cells (Table 1.)

TABLE 1
Comparative Sensitivity Characteristics of Cultures of HeLa-2 Tumor
Cells to Composition

| — | | Composition Activity at Various Acidity | |
|---|---|---|---|
| | MAD in | Levels among 199 ¹ Cell Cultures. | |
| Drug | mcg/ml | Control | Experiment |
| Composition | 10 | 0 | ++++ |
| Taxotere | 10 | 0 | ++ |
| Bare | — | 0 | 0 |
| liposomes | | | |

¹ Cytopathic activity;
++++—degeneration of 100% of the cells
0 —lack of degeneration.

In establishing the minimum concentration of Composition that will slow the growth of cells, a comparison was made between the number of surviving cells and the concentration of Composition in the solution.

TABLE 2
The Effect of Composition on HeLa Cells

| | Number of | | | | |
|---|---|---|---|---|---|
| | cells | Number of Live Cells | | Number of Live Cells | |
| | before | after Incubation, | | after Incubation, | |
| Dose, | Incubation, | Millions, ± 1000 | | Millions, % | |
| mcg/ml | Millions | Composition | Taxotere | Composition | Taxotere |
| 2 | 150000 ± 1000 | 72000 | 150000 | 48 | 100 |
| 4 | 153000 ± 1000 | 21400 | 150000 | 14 | 98 |
| 6 | 150000 ± 1200 | 9800 | 145000 | 6.5 | 97 |
| 8 | 152000 ± 1000 | 0 | 135000 | 0 | 89 |
| 10 | 158000 ± 1000 | 0 | 130000 | 0 | 82 |
| 12 | 162000 ± 1000 | 0 | 153000 | 0 | 94 |

As may be seen in Table 2, an effective dose of Composition is between 8-12 mcg/ml solution.

Composition led to a 95% degeneration of tumor cells. To confirm the in vivo antitumor activity, Composition was studied in benzidine skin sarcoma and reinjected ascites adenocarcinomas in Barbados mice. In five mice with adenocarcinomas, the distribution of the Composition liposome throughout the animals' bodies was also studied using a fluorescent probe dissolved in a phospholipid layer.

A Study of the Anticancer Activity of Composition on Benzidine Sarcoma. Before applying it to the silica gel, 7 ml of a solution of 2% benzidine and 0.9% sodium chloride was added until an opalescent suspension was formed (1 g silica gel for 5 ml NaCl solution). Twenty-five Barbados mice of both sexes with a weight of 18-20 g that were kept on a vivarium diet were administered benzidine and phorbol acetate immobilized on silica gel subcutaneously near the neck. After two weeks, 18 animals had developed tumors of different sizes in the form of a small bump on the neck near the silica gel granulomas. Each group of animals was administered the corresponding compound parenterally at a dose of 100 mcg/kg weight twice a day for two weeks, beginning at 16 days after administration of the carcinogen.

TABLE 3
A Comparison of the Antitumor Activity of Composition in
Comparison to the Combination of an Taxotere and a Lipid

| | Weight of Animal (g) | |
|---|---|---|
| Drug Name | Before Treatment | After Treatment |
| Taxotere | 28 ± 1.2 | 23 ± 1.1 (2 mice died) |
| Composition | 25 ± 1.7 | 15 ± 1.5 |
| Bare Liposomes | 26 ± 2.1 | 34 ± 1.3 (5 mice died) |

Note:
n = 7, p > 0.05 in comparison with the control and previous data.

As may be seen in Table 3, Composition decreased the weight of the experimental animals by 10 g; the control animals' weight continued to increase, and some of them died. After the dissection of the silica gel granulomas, it was established that the animals treated with Composition did not show signs that the granulomas had turned into malignant sarcomas.

The animals' survival rates are presented in Table 4.

TABLE 4
Survival Rates of Animals with Benzidine Skin Sarcoma

| Drug Name | Animal Survival, Days |
|---|---|
| Taxotere | 28 ± 1.1 |
| Composition | 49 ± 1.2 |
| Bare Liposomes | 17 ± 0.9 |

Note:
n = 10, p > 0.05 in comparison with the control and previous data.

Thus Composition prolongs the life of animals twice as long as does Taxotere.

A Study of the Antitumor Activity of Composition on Ehrlich's Ascites Adenocarcinoma

The antitumor activity of the compositions were studied in models of Ehrlich's ascites carcinoma in young Barbados mice of both sexes with weights between 15-17 g (68 individuals), which were kept on a vivarium diet.

45 mice were inoculated from a mouse with adenocarcinoma using an insulin syringe with 0.1 ml ascitic fluid in the region of the liver. Within seven days, 42 mice showed signs of tumors (the body weight and belly size increased); two mice died on the second day; one mouse did not show signs of a tumor.

Ten mice were administered Composition in liposome form (see Table 5). Ten more mice were administered the Composition substance, and ten more were administered a 0.9% solution of sodium chloride with lipids.

TABLE 5
Qualitative Biological and Statistical Characteristics in the Study of
Composition's Antitumor Activity

| | | Time of Death | |
|---|---|---|---|
| | | of Animals after the First Injection | |
| | | Average Value, Days | |
| Substance | Liposomes | Experimental Animals | Control Animals |
| Composition | + | 37.4 ± 0.88 | 3.2 ± 0.44 |
| -//- | − | 18 ± 3.2 | 3.1 ± 0.48 |
| Taxotere | + | 15 ± 0.5 | 3 ± 0.5 |
| -//- | − | 14 ± 0.12 | 3 ± 0.6 |

Note:
n = 10, p > 0.05 in comparison with the control and previous data.

Composition was given to those mice from which blood was drawn. Mice with Ehrlich's adenocarcinoma, after being given the tumor and treated, lived for 18 days when administered the Composition substance, which is 6 times longer than the control, and 37 days when administered Composition in liposomes, which is 12 times longer than the control. At an accuracy level of more than 99.5%, we can confirm a significant increase in anticancer activity in liposomal Composition over the control, Taxotere. After dissection of the animals, signs of tumors and metastasis were not found in their bodies.

1. A pharmaceutical composition, comprising a mixture of carboxylated oligonucleotides, obtained by hydrolysis of natural polynucleotides, that resulted in a oligonucleotide mixture, and carboxylation of purine nucleotide bases in the oligonucleotide mixture.

2. The pharmaceutical composition of claim 1, wherein the natural polynucleotide is a whole RNA of eukaryotic cell.

3. The pharmaceutical composition of claim 1, wherein the natural polynucleotide is a whole DNA of eukaryotic cell.

4. The pharmaceutical composition of claim 1, wherein the hydrolysis of the natural polynucleotides is provided by nuclease.

5. The pharmaceutical composition of claim 1, wherein the hydrolysis of the natural polynucleotides is provided by synthetic nuclease.

6. The pharmaceutical composition of claim 1, wherein the hydrolysis of the natural polynucleotides is provided by acid or alkaline hydrolysis.

7. The pharmaceutical composition of claim 1, wherein the carboxylation of the oligonucleotides is provided by acylation with succinic anhydride.

8. The pharmaceutical composition of claim 1, wherein the carboxylation of the oligonucleotides is provided by alkylation with monochloroacetic acid.

9. The pharmaceutical composition of claim 1, wherein the carboxylation of purine nucleotide bases is calculated through a mass modifier (Mm) in a weighed portion of a dry mixture of oligonucleotides (Mo):
$$Mm = {M}_{r}^{m} ⌊ \frac{n {2}^{( n - 1 )}}{( {2}^{n} - 1 )} ⌋ , \qquad ( 1 )$$
where
n—Quantity of the purine bases, available for modification of the taken weighed portion of dry oligonucleotide composition,
Mᵣ^{m}—molecular weight of the modifier, g/mol
n—is calculated by the formula:
$$n = ( \frac{{M}_{o} - {m}_{u} - {m}_{c} - {m}_{a}}{{M}_{r}^{G}} ) + ( \frac{{M}_{o} - {m}_{u} - {m}_{c} - {m}_{g}}{{M}_{r}^{A}} ) , \qquad ( 2 )$$
where
Mo—portion weight of the dry mixture of oligonucleotides after hydrolysis, g;
mᵤ—uridyl weight in the portion of the dry mixture of oligonucleotides after hydrolysis, identified by chromatography after complete hydrolysis, g;
m_{c}—cytosyl weight in the portion of the dry mixture of oligonucleotides after hydrolysis, identified by chromatography after complete hydrolysis, g;
mₐ—adenosyl weight in the portion of the dry mixture of oligonucleotides after hydrolysis, identified by chromatography after complete hydrolysis, g;
m_{g}—guanosyl weight in the portion of the dry mixture of oligonucleotides after hydrolysis, identified by chromatography after complete hydrolysis, g;
M_{T}^{G}—molecular weight of guanosyl, g/mol;
M_{T}^{A}—molecular weight of adenosyl, g/mol.
